(19) United States
(12) Plant Patent Application Publication
(10) Pub. No.: US 2009/0193556 P1
(43) Pub. Date: Jul. 30, 2009

(54) Anigozanthos hybrid plant named 'Regal Velvet'

(50) ;

(76) Inventor: Keith Oliver, Hamersley (AU)

Correspondence Address: Sam R. McCoy Jr., P.O. Box 2108, Mount Pleasant, SC, 29465, US

(21) Appl. No.: 12/011,409

(22) Filed: Jan. 25, 2008

(51) Int. Cl. A01H 5/00

(52) U.S. Cl. PLT/263.1

(57) ABSTRACT

‘REGAL VELVET’ is a distinctive variety of Anigozanthos hybrid which is characterized by the combination of its medium plant height with an upright growth habit and a semi-erect leaf attitude, large number of flowers per inflorescence and a flower color consisting of yellow green colored perianth tube with a red colored ovary.

### LATIN NAME OF THE GENUS AND SPECIES

The Latin name of the genus and species of the novel variety disclosed herein is Anigozanthos hybrid.

### VARIETY DENOMINATION

The inventive variety of Anigozanthos disclosed herein has been given the variety denomination ‘REGAL VELVET’.

### BACKGROUND OF THE INVENTION

The present invention relates to a new and distinct perennial variety of Anigozanthos hybrid, which has been given the variety denomination of ‘REGAL VELVET’. Its market class is that of an ornamental plant. ‘REGAL VELVET’ is intended for use in landscaping and as a decorative plant.

The Anigozanthos hybrid variety ‘REGAL VELVET’ was the result of a controlled pollination between seed parent un-named Anigozanthos manglesii and pollen parent un-named Anigozanthos flavidus in 2002 in an Australian nursery in the state of Western Australia. Seed were collected and germinated in vitro during 2003. Resultant plants were tested in 200 mm pots and in ground during 2003-2004. The inventive variety was finally selected in 2004 based on strong plant growth vigor and flowering traits.

‘REGAL VELVET’ is propagated asexually by micropropagation at a tissue culture laboratory in Tumbi Umbi in the state of New South Wales, Australia. The distinctive characteristics of the inventive ‘REGAL VELVET’ variety are stable from generation to generation; clones of the variety produced by asexual reproduction maintain the distinguishing characteristics of the original plant.

‘REGAL VELVET’ has a medium plant height including the inflorescence (range 90-120 cm) with an upright growth habit and a semi-erect leaf attitude whereas the seed parent has a short plant height (range 60-90 cm) and the pollen parent has a tall plant height (150-180 cm). ‘REGAL VELVET’ has a large number of flowers per inflorescence with the predominant color of the perianth tube being yellow-green whereas the seed parent has a green predominant perianth tube color and the pollen parent has a greenish to red (with age) predominant perianth tube. Time of flowering for ‘REGAL VELVET’ is spring whereas the seed parent is late winter to spring and the pollen parent is late spring to summer.

An application for plant breeders' rights for variety ‘REGAL VELVET’ has been lodged with the Australian Plant Breeders' Rights Office, and was first gazetted in February 2006 under Application No. 2006/012.

### SUMMARY OF THE INVENTION

‘REGAL VELVET’ is a distinctive variety of Anigozanthos hybrid which is characterized by the combination of its medium plant height with an upright growth habit and a semi-erect leaf attitude, large number of flowers per inflorescence and a flower color consisting of yellow green colored perianth tube with a red colored ovary.

### BRIEF DESCRIPTION OF THE FIGURES

FIG. 1 shows a ‘REGAL VELVET’ plant.

FIG. 2 shows a ‘REGAL VELVET’ plant foliage.

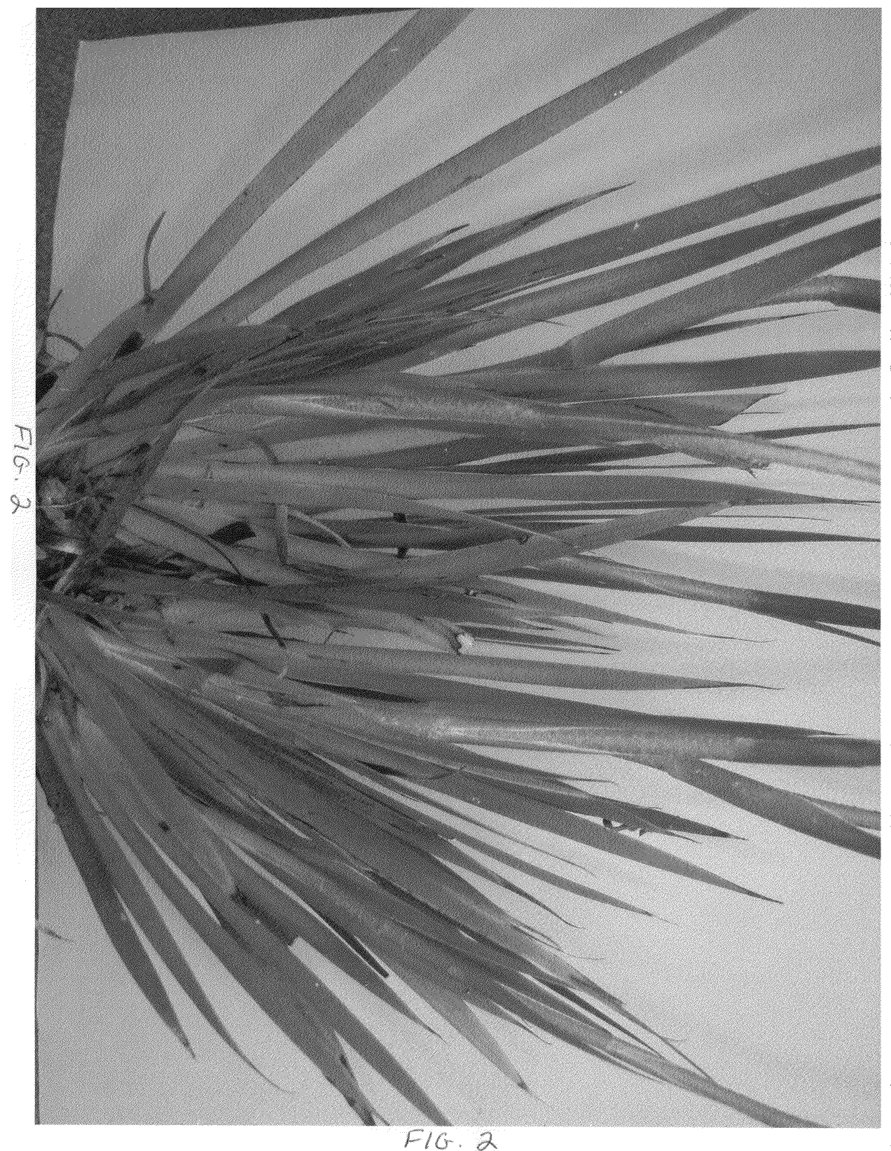

FIG. 3 shows a ‘REGAL VELVET’ inflorescence branching pattern.

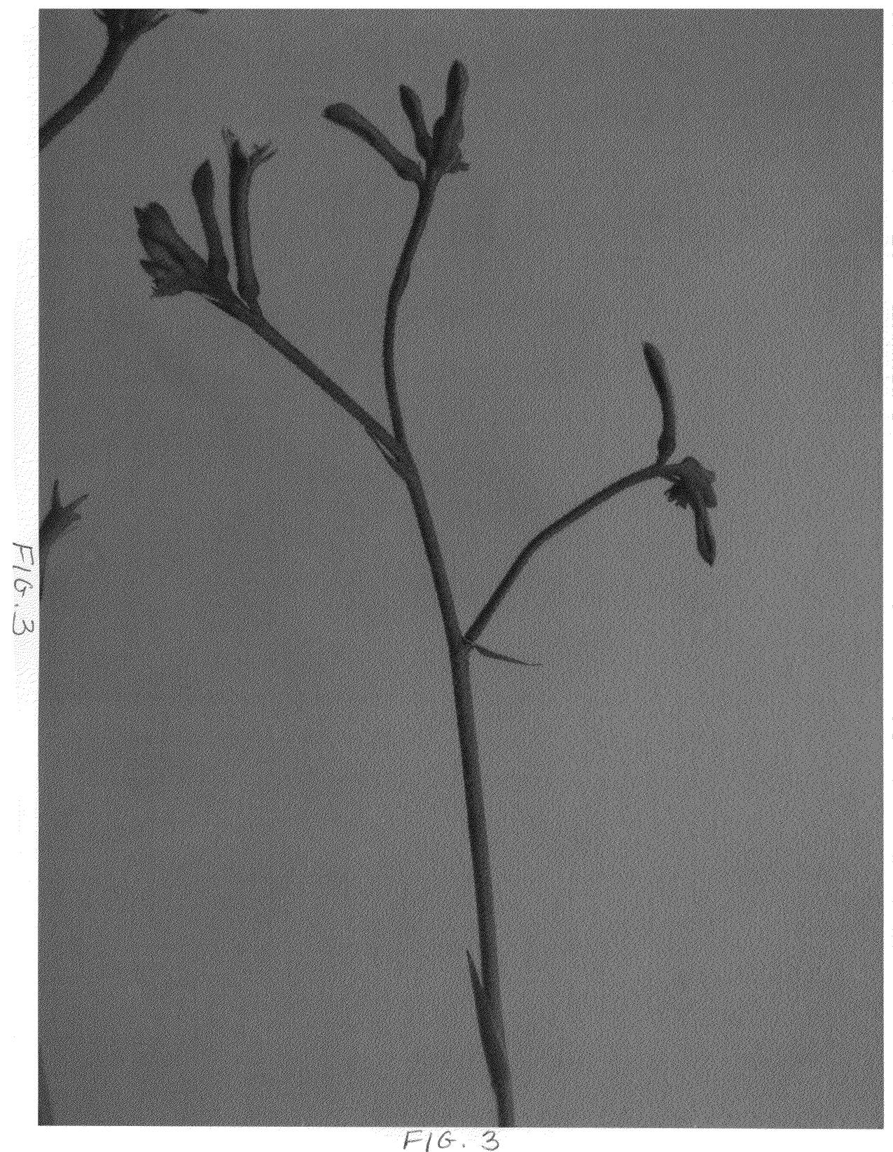

FIG. 4 shows ‘REGAL VELVET’ flowers within the inflorescence.

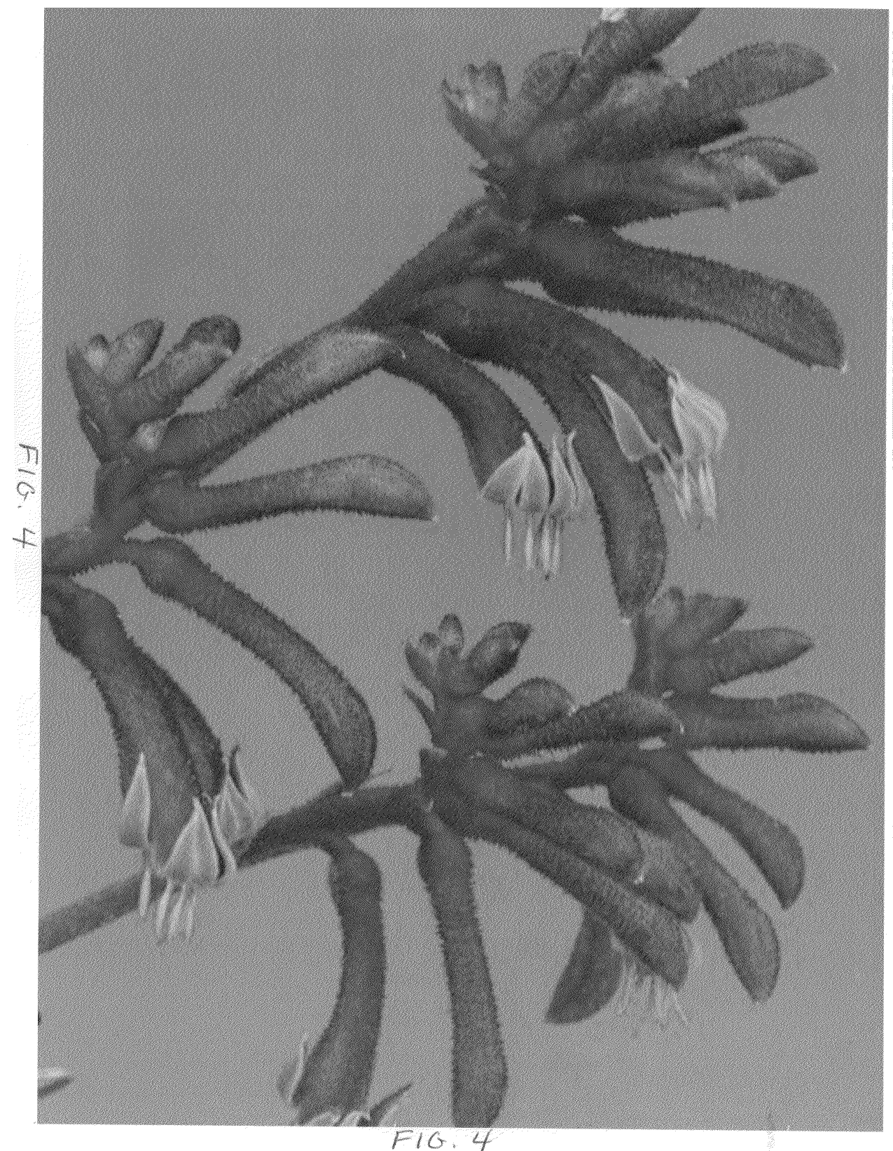

FIG. 5 shows ‘REGAL VELVET’ flower outer perianth tube and ovary.

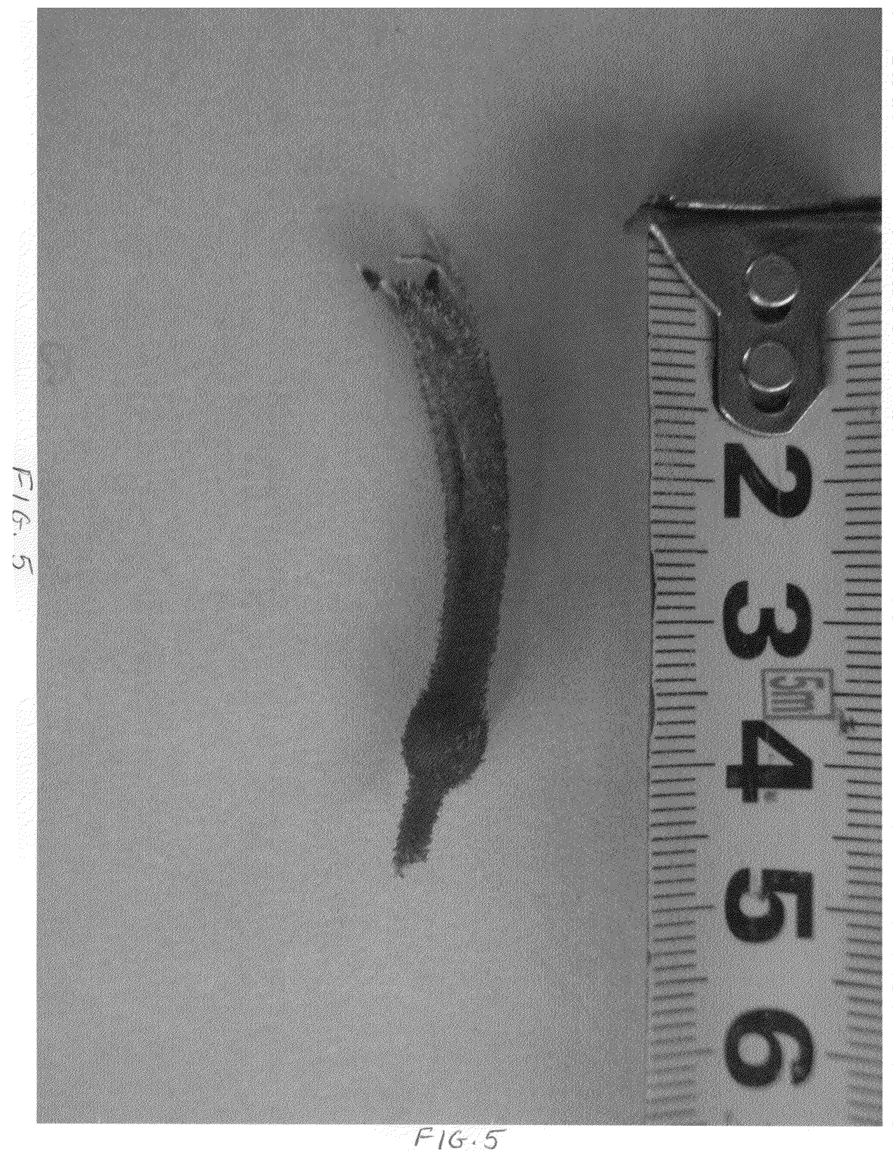

FIG. 6 shows ‘REGAL VELVET’ anther position detail.

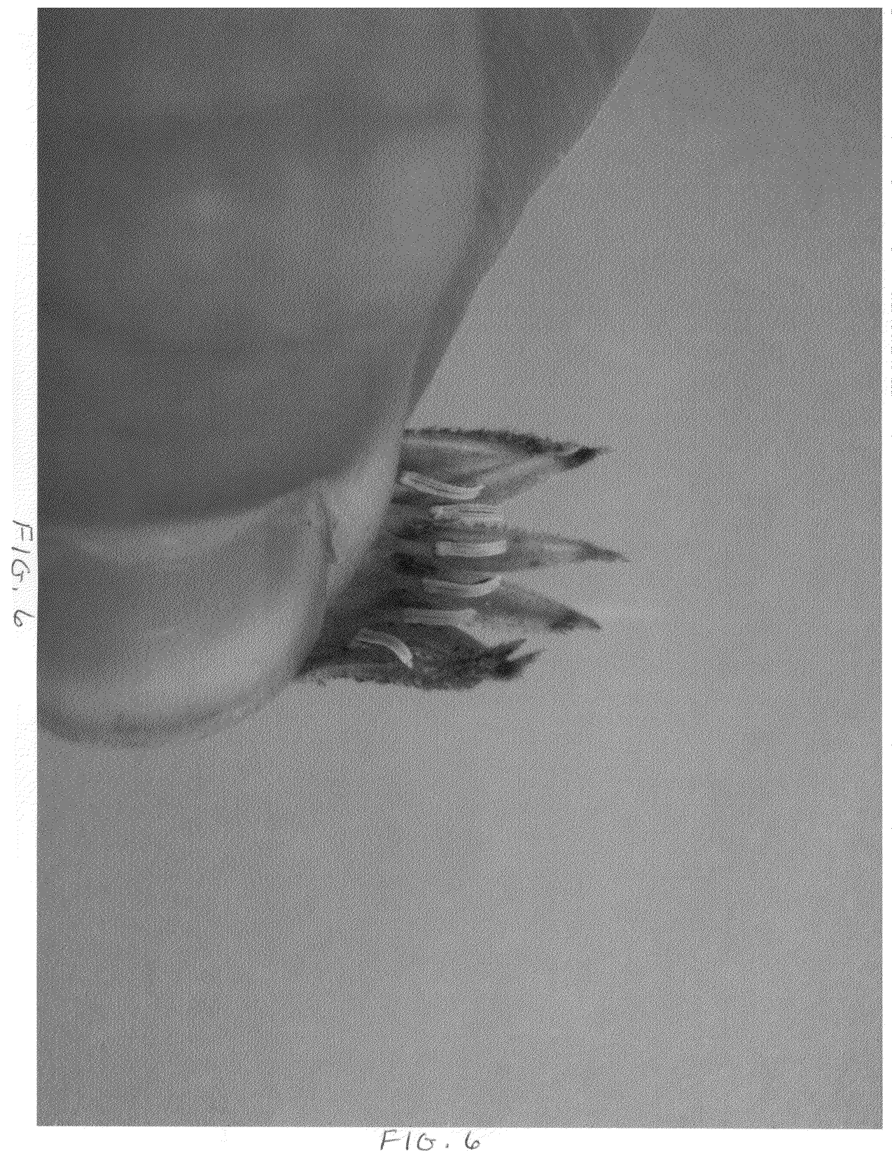

FIG. 7 shows ‘REGAL VELVET’ anther position with respect to stigma.

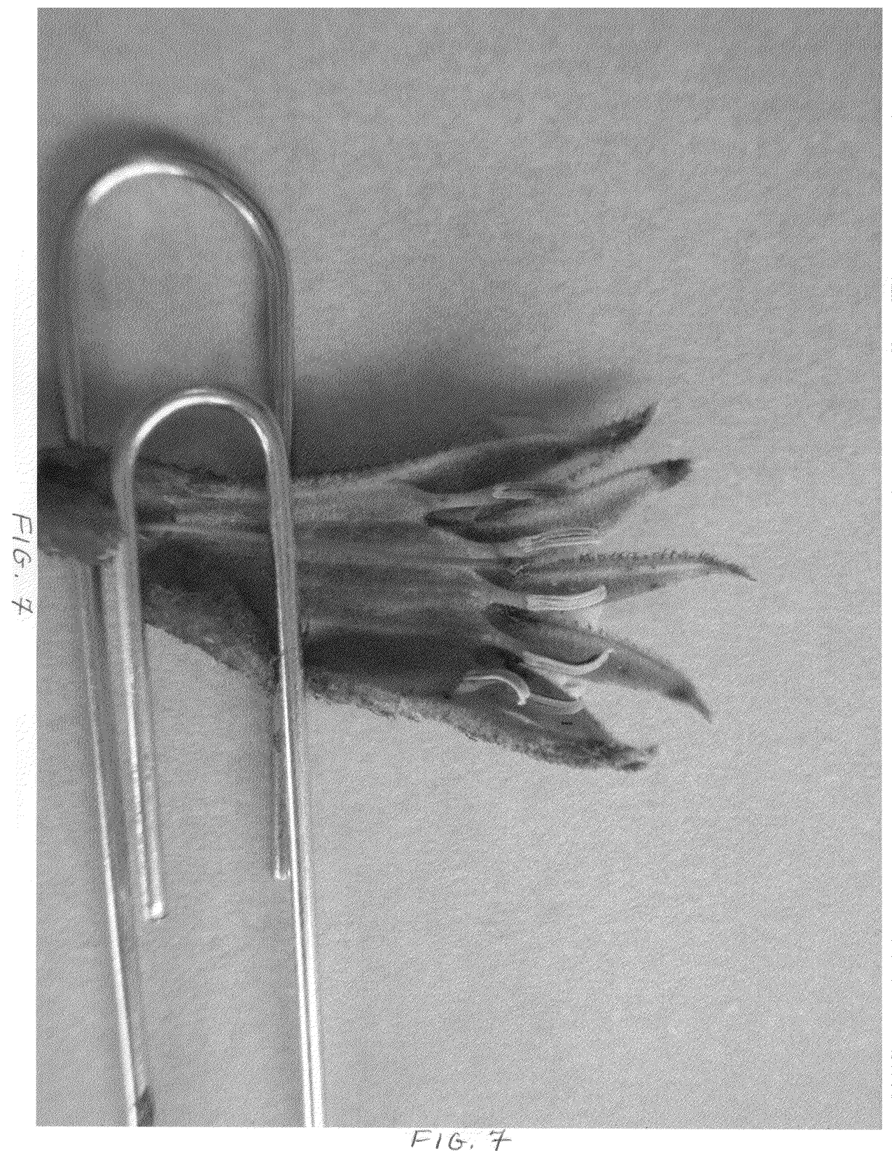

### BOTANICAL DESCRIPTION OF THE PLANT

The following is a detailed botanical description of a new and distinct variety of an Anigozanthos hybrid ornamental plant known as ‘REGAL VELVET’. Plant observations were made on plants, 6 months old, grown in New South Wales, Australia. Unless indicated otherwise, the descriptions disclosed herein are based upon observations made from October 2006 of mature ‘REGAL VELVET’ plants grown in nursery pots.

Those skilled in the art will appreciate that certain characteristics will vary with older or, conversely, younger plants. ‘REGAL VELVET’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Color Chart, The Royal Horticultural Society, London, 1995 edition. ‘REGAL VELVET’ is a perennial Anigozanthos hybrid plant which resulted from a controlled pollination between seed parent un-named Anigozanthos manglesii and pollen parent un-named Anigozanthos flavidus. After its selection, ‘REGAL VELVET’ was asexually propagated by micropropagation. ‘REGAL VELVET’ has a medium plant height including the inflorescence with an upright growth habit and a semi-erect leaf attitude, large number of flowers per inflorescence and a predominantly yellow green colored perianth tube with change to red color towards the ovary.

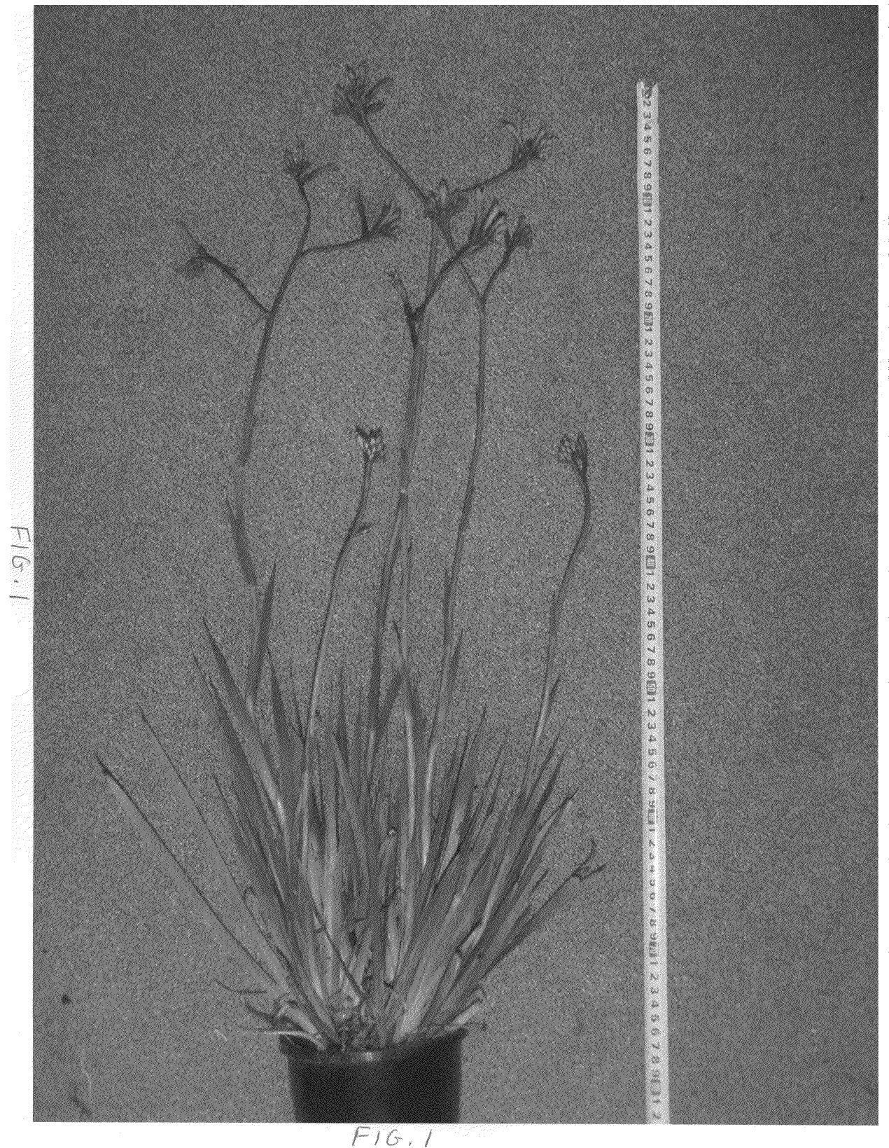

These features and other characteristics are apparent from the description provided below.
Growth habit, dimensions and color: ‘REGAL VELVET’ is a medium, rhizomatous plant forming an upright fan shaped tuft. Average plant height including the inflorescence is about 100 cm (range 90-120 cm), average height of foliage only is 40 cm (range 35-50 cm) and average plant spread is 40 cm in a mature plant grown in nursery or field plots in Sydney, New South Wales, Australia (observed spring 2006). The number of inflorescences is medium. The mature leaf length is medium (range 25-35 cm) and the mature leaf width is medium (approximately 14-16 mm in a 140 mm pot). The leaf attitude is semi-erect and the degree of leaf curvature is slightly curved to straight. The upper and lower sides of the leaf are yellow green (approximately RHS 147A) in color (observed spring 2006). Leaf glaucosity is very weak. The degree of hairiness of the leaf margin is absent to very weakly expressed. The leaf shape is linear-slightly falcate, leaf base is cauline, leaf margin is entire, leaf apex is acute and leaf surface texture is glabrous. The leaf venation pattern is parallel; the color is the same as the rest of the leaf. The leaf attitude is semi-erect. A representative ‘REGAL VELVET’ plant is shown in FIG. 1.
Roots: The roots of ‘REGAL VELVET’ are fibrous, freely branching and white in color and are similar to other Anigozanthos hybrids.
Rhizomes: The rhizomes of ‘REGAL VELVET’ are short creating a tufted plant similar to other Anigozanthos hybrids.
Inflorescence: Branching of the inflorescence is present and the degree of ramification is secondary. The length of the lowest lateral is short-medium (approximately 8 to 12 cm). The number of flowers per inflorescence is medium, a typical plant having 2-3 racemes per inflorescence, each with about 9 flowers (longer than 3 mm) in a 140 mm pot size (observed spring 2006). Peduncle diameter at the base is approximately 9 mm. The peduncle length averages 100 cm (range 90-110 cm), Color of peduncle is approximately yellow green RHS 146B with pubescence approximately grayed purple 187A-B.
Flowers: Bud shape is ovate to oval in longitudinal section and round in cross section; bud length is 7-10 mm ; bud diameter is 5-6 mm ; bud apex is broadly acute; bud base is obtuse to rounded; bud color is yellow green RHS 146C-D with dense pubescence approximating to red RHS 46A. Pedicel length is 4-5 mm; pedicel diameter is 2.5-3.0 mm. The color of hairs on the pedicel corresponds to red (approximately RHS 46A). Anigozanthos do not have petals. Instead they have a perianth tube with lobes (length 11 mm, width 1.5-2.5 mm); lobe shape is deltoid; apex is acute, margin entire; The perianth tube length is medium (range 32 to 50 mm) and the width is medium (approximately 7 mm below perianth lobes) on an opening flower. Perianth tube profile is broadening evenly. The predominant color of the outer perianth tube is yellow green (approximately RHS 146C). The number of colors on hairs on the perianth tube is one and this corresponds to red (approximately RHS 46A). The inner color of the perianth tube is a yellow green (RHS 146C). The inner color of the perianth tube lobes is a grayed green (RHS 191A-B); meaning that the color falls somewhere in between the two colorations. Perianth lobe length is medium (approximately 11 mm for the longest lobe). Reflexing of the lobes is medium to strong. The number of anthers at the top of the perianth is four. The color of the hairs on the ovary corresponds to red (approximately RHS 46A). The position of the stigma in relation to the anthers is level. Time to beginning of flowering is early-medium. Pollen production is scarce; color approximately yellow white RHS 159D. No fragrance observed. Floral bract length: up to 7 cm. Width: About 6 mm at base. Shape: Ensiform tapering to a narrow conduplicate form at the apex. Apex: narrow acute. Base: Clasping. Margin: Entire. Texture, upper surface: Smooth, glabrous. Texture, lower surface: tomentose. Color, upper surface: 137B. Color, lower surface: 137B. Reproductive organs: stamen number is 6; anther shape is linear with occasional curvature; anther length is approximately 4 mm; anther width is 0.7 to 1.0 mm ; anther color is approximately in the range 159C-D. Pistil number is 1; pistil length is approximately 28-30 mm; style length is 26-28 mm; style color is approximately yellow green RHS 144B. Stigma shape is trullate with stigma color yellow green RHS 144B. Ovary color is approximately yellow green RHS 144B.
Comparison of ‘Regal Velvet’ with other varieties of Anigozanthos hybrid: ‘REGAL VELVET’ combines a medium plant height with an upright growth habit and a semi-erect leaf attitude, large number of flowers per inflorescence and a predominantly yellow green colored perianth tube with change to red color towards the ovary compared to other Anigozanthos hybrids. ‘REGAL VELVET’ has a shorter plant height including the inflorescence than another comparable type known as ‘Big Red’ (unpatented) with a height of 90-120 cm whereas ‘Big Red’ is 150-180 cm tall. ‘REGAL VELVET’ has a predominantly medium yellow green perianth tube color due to a relatively low density of red colored hairs present whereas ‘Big Red’ has a predominantly red perianth tube color due to a much higher density of red colored hairs. ‘REGAL VELVET’ has two predominant flower colors created by the contrast between a yellow green perianth tube and a red ovary where the red hairs are dense relative to the perianth tube whereas ‘Big Red’ has a single red flower color due to the higher density of red colored hairs over the perianth tube and the ovary. ‘REGAL VELVET’ has a spring flowering season whereas ‘Big Red’ has a spring to summer flowering season. The combination of medium plant height with an upright growth habit and a semi-erect leaf attitude, large number of flowers per inflorescence and a predominantly yellow green colored perianth tube with change to red color towards the ovary makes ‘REGAL VELVET’ a desirable ornamental plant suited for mass production for pot and landscape use.
Asexual reproduction: ‘REGAL VELVET’ was germinated as a seedling in vitro and asexually reproduced by micropropagation. Several generations have been reproduced and ‘REGAL VELVET’ has been observed to retain all distinguishing characteristics that were noted in the original ‘REGAL VELVET’ seedling. In vitro plants of ‘REGAL VELVET’ initiate roots in 7 to 10 days.
Environmental tolerances: ‘REGAL VELVET’ plants have exhibited good tolerance to rain and wind and to tolerate temperatures from 1 to about 40 degrees Celsius. ‘REGAL VELVET’ has moderate to good drought tolerance. ‘REGAL VELVET’ has moderate to good recovery with watering after severe wilting. ‘REGAL VELVET’ does well in sandy and gravelly soils.
Disease resistance: ‘REGAL VELVET’ has not been observed to be resistant to other pathogens and pests common to Angiozanthos. Further testing is underway.

That which is claimed is:

1. A new and distinct variety of Anigozanthos hybrid plant named ‘REGAL VELVET’, substantially as described and illustrated herein.
